(19) United States
(12) Patent Application Publication
(10) Pub. No.: US 2010/0152114 A1
(43) Pub. Date: Jun. 17, 2010

(54) Antioxidant activity of GH-RH Antagonists

(75) Inventors: Andrew V. Schally, (US); Nektarios Barabutis, (US)

Correspondence Address: OMRI M. BEHR, 325 PIERSON AVENUE, EDISON, NJ, 08837-3123, US

(73) Assignee: Univ of Miami and USA by Dept of Veterans Affairs

(21) Appl. No.: 12/626,913

(22) Filed: Nov. 29, 2009

Related U.S. Application Data
(60) Provisional application No. 61/122,171, filed on Dec. 12, 2008.

(51) Int. Cl. A61K 38/16; A61P 35/00

(52) U.S. Cl. 514/12

(57) ABSTRACT

There are provided means for suppressing the Reactive Oxidant Species (ROS) of certain cells by the administration of GHRH antagonists. The therapeutic applications of the anti-oxidative action of GH-RH antagonists relate to the redox status of certain cells, including but not limited to cancer cells, reducing the metabolism of reactive oxygen and nitrogen species. This antioxidant activity of GHRH antagonists is employable in the treatment of diseases in which their pathogenesis is related to increased cellular level of oxidative stress.

### RELATED APPLICATIONS

This application claims priority of applicants' copending provisional application Ser. No. 61/122,171 filed Dec. 12, 2008.

This invention was made in part with Government support from the Medical Research Service of the Veterans Affairs Department. The Government has certain rights in this application.

### FIELD OF INVENTION

There are provided means for suppressing the Reactive Oxidant Species (ROS) of the redox status of certain cells, including but not limited to cancer cells, reducing the metabolism of reactive oxygen and nitrogen species. This antioxidant activity of GHRH antagonists is employable in the treatment of diseases in which their pathogenesis is related to increased cellular level of oxidative stress.

### DISCUSSION OF THE PRIOR ART

The development of antagonistic analogs of Growth Hormone-Releasing Hormone (GHRH) started more than a decade ago. GH-RH neuropeptide, secreted by the hypothalamus, regulates the release of Growth Hormone from the anterior pituitary gland. GHRH was first isolated from human pancreatic tumors and only subsequently identified in human hypothalamus.

The fact that GHRH is implicated as a growth factor in carcinogenesis was established only recently although its initial identification from tumor tissue should have provided a hint about this likelihood. Thus the expression of mRNA for GHRH and the presence of biologically active GHRH were demonstrated in several established cancer cell lines and human tumors. The suppression of proliferation of breast, prostate and lung cancer cell lines after the knocking down of the GHRH gene expression supports the concept that GHRH functions as growth factor at least in these human cancers. Peptide receptors that mediate the effects of GHRH and its antagonists on tumors were also identified recently with the demonstration that cancers can express splice variants (SVs) of the pituitary GHRH receptor (pGHRH-R) as well as the pituitary type itself.

Several series of antagonistic analogues of GHRH have been synthesized and have shown that they inhibit the growth of a variety of experimental human cancers The inhibitory effect of antagonistic analogs of GHRH is exerted in part by endocrine mechanisms through the suppression of GHRH-evoked GH release from the pituitary, which, in turn results in the reduction of hepatic IGF-I levels in serum. The anti-tumor effects of GHRH antagonists can be also exerted directly on tumors and based upon the blockade of action of autocrine GHRH in tumours as well as the inhibition of the secretion of autocrine/paracrine IGF-I or IGF-II from the tumors.

The influence of the GHRH analogs in the redox (reduction/oxidation) status of cancers has not been previously investigated. The central role in redox signaling is played by the reactive oxygen species (ROS) which are oxygen radicals and non radical derivatives of O₂, thus highly reactive molecules. When organic radicals are generated within an organism they can react rapidly with DNA, proteins, and lipids causing chemical modification, collectively known as oxidative stress. ROS are produced continuously by the mitochondria, macrophaghes and peroxisomes.

Reactions between ROS and redox active amino acid residues in transcription factors and enzymes can modulate the activities of these proteins. Cells possess effective mechanisms to control ROS. Among these is the synthesis of detoxifying enzymes such as thioredoxins (Trxs), superoxide dismutases (SODs) glutathione peroxidases (GPxs) and quinone oxidoreductase 1 (NQ01), which convert ROS into less active

ROS and cellular oxidant stress are associated with cancer in a complex fashion .species (.Schumacker PT Reactive oxygen species in cancer cells: live by the sword, die by the sword Cancer Cell 2006, 10(3): 175-176). Cancer cells produce more ROS than normal cells and ROS are thought to play a role in tumor initiation and progression and are also required for aggressive phenotype. (Kumar B, Koul S, Khandrilka L, Meacham R B, Doul H K Oxidative stress is inheritent in prostate cancer cells and is required for aggressive phenotype. Cancer Res 68: 1777-1785 (2008)). Abnormal increases in ROS can be exploited to selectively kill cancer cells. Exogenous ROS stressing agents can increase the intracellular ROS to a toxic level, or the threshold that triggers cell death.

The wild-type tumor suppressor protein p53 which is expressed in LNCaP cells acts as a major defense against cancer and can elicit apoptotic death, cell cycle arrest or senescence through differential activation of target genes in order to maintain the genomic integrity. Given that both ROS and P53 participate in multiple cellular processes, the interactions between them and their signaling pathways should exist Liu B, Chen Y, St Clair D K ROS and p53: A versatile partnership. Free Radic Biol Med 44:1529-1535 (2008)). The induction of the expression of the wild type p53 is related to antioxidant activities which also contribute to tumor suppression (Sablina A A, et al. The antioxidant function of the p53 tumor suppressor. Nat Med 11:1306-1313 (2005)). We examined whether the expression of the wild type p53 is influenced by treatment with GHRH antagonist and GHRH(1-29)NH₂.

Activation of the MAPK signaling pathway, which is stimulated by GHRH (Pombo C M, Zalvide J, Gaylinn B D, Dieguez C Growth Hormone Releasing hormone stimulates mitogen-activated protein kinase. Endocrinology 141:2113-2119 (2000)) is implicated in the progression of tumorigenesis (H. Dolado I, et al. p38alpha MAP kinase as a sensor of reactive oxygen species in tumorigenesis. Cancer Cell 11:191-205 (2007)). Wild type p53 suppresses the promoter of the PCNA in order to mediate DNA synthesis and repair processes In addition, PCNA can play a critical role in regulating the stability of p53. The inactivation of PCNA can induce stabilization of p53.

+

Previous studies which supported the antiapoptotic role of GHRH in cancer cells and the induction of apoptosis by GHRH antagonists in tumors. In addition inhibition of the MAPK pathway enhances apoptotic death. The activation of the NF-κB p50 which promotes carcinogenesis is enhanced by oxidative stress (Bar-Shai M, Carmen E, Ljubuncic P, Reznic A Z Exercise and immobilization in aging animals: The involvement of oxidative stress and NFKappaB activation. Free Radic Biol Med 44:202-214 (2008)).

### SUMMARY OF THE INVENTION

The expression of the GHRH-R and its splice variant 1 in a cancer cell line has been studied, as well as the effect of GHRH (1-29) NH₂ and GHRH antagonist JMR-132 ([PhAc⁰-Tyr¹, D-Arg², Cpa⁶, Ala⁸, Har⁹, Tyr(Me)¹⁰, His¹¹, Abu¹⁵, His²⁰, Nle²⁷, D-Arg²⁸, Har²⁹]hGH-RH(1-29)NH₂) on the proliferation rate of cells and on the expression of the proliferating cell nuclear antigen (PCNA). We examined the expression of the wild-type tumor suppressor protein p53, the transcription factor NFκB p50 and its phosphorylated form as well as the caspase 3 and the cleaved caspase 3 which act on apoptosis.

The GHRH(1-29)NH₂ and the GHRH antagonist influence on the expression of the antioxidant enzymes, superoxide dismutase (SOD1) which is also a target for the inhibition of angiogenesis and tumor growth, quinone oxidoreductase 1 (NQ01), a cytosolic protein that reduces and detoxifies quinones protecting the cells against redox cycling and oxidative stress, GPX1 which is a main glutathione peroxidase and thioredoxin 1 (Trx1), a major cytoplasmic antioxidant enzyme [31] were examined. In addition, the influence of GHRH and GHRH antagonist on the expression of cyclooxygenase 2 and cytochrome c oxidase IV, enzymes that are involved in the generation of the ROS, has been reviewed.

In order to elucidate the oxidative status of the cancer cell line before and after treatment with the GHRH antagonist. The expression of the 3-nitrotyrosine and the protein carbonyl groups which are considered as markers of protein oxidative modifications (Dane Donne I, Rossi R Giustarini D, Mizani A, Colombo R (2003) Protein carbonyl groups as blomarkers of oxidative stress, Clin Chim Acta 329:23-38) as well as the malondialdehyde (MDA) which reflects the status of the lipid peroxidation were studied, as well as the influence of the GHRH and the JMR-132 on the intracellular generation of the reactive oxygen species.

The antioxidant activity of Growth Hormone Releasing Hormone (GHRH) Antagonists influences the redox status of certain cells, including but not limited to cancer cells, reducing the metabolism of reactive oxygen and nitrogen species. This antioxidant activity of GHRH antagonists is employable in the treatment of diseases in which their pathogenesis is related to increased cellular level of oxidative stress. This increase can be related to dysfunction of the natural antioxidative mechanisms as well as defective mitochondrial function. This is of utility with respect to all the neurodegenerative disorders, like Alzheimer's and Parkinson's disease, amyotrophic lateral sclerosis, Huntington's and Alexander's disease as well as inherited ataxias. The redox cellular abnormalities which can also result to cellular protein and lipid damage, are also involved in diabetes and its complications, like macro- and micro-vascular disorders as well as the diabetic neuropathy and diabetic neuropathy.

The present invention is not limited to particular GH-RH antagonists. Any compound in this category may be used. It is desirable to utilize peptide GHRH antagonists, in particular those of high antagonistic activity and in particular affinity for cancer cells. Thus, of substantial utility, are the highly antagonistic peptides disclosed in PCT application PCT/US09/38351 and pending application Ser. No. 12/562,010 and Ser. No. 12/562,096 whose disclosure is Incorporated herein by reference.

### BRIEF DESCRIPTION OF THE DRAWINGS

FIG. 1: shows the effect of GHRH and JMR-132 on the proliferation rate of the LNCaP cancer cell line measured after an incubation of 72 hours.

(A) is a graphic presentation of the changes in the proliferation rate of the LNCaP prostate cancer cell line after exposure to GHRH antagonist JMR-132 and GHRH (1-29)NH₂. Percentage increase or decrease are expressed vs LNCaP cells cultured in the absence of JMR-132 or GHRH (1-29)NH₂*(P<0.05), **(P<0.005).

(B) is a Western Blot analysis of expression of the Proliferating Cell Nuclear Antigen (PCNA) in LNCaP prostate cancer cell line after exposure to GHRH antagonist JMR-132, and GHRH(1-29)NH₂ n=2.

FIG. 2: shows a Western Blot analysis of expression of the wild p53 tumor suppressor protein in LNCaP prostate cancer cell line after 72 hour exposure to GHRH antagonist JMR-132 and GHRH(1-29)NH₂. n=2

FIG. 3: shows the effect of GHRH and JMR-132 on the activation of caspase 3 and NFκB p50 measured after an incubation of 72 hours.

(A): is a Western Blot analysis of expression of the phosphorylated NFκB p50, caspase 3 protein and its cleaved form in LNCaP prostate cancer cell line after exposure to GHRH antagonist JMR-132 and GHRH (1-29)NH₂. n=2

(B): is a Western Blot analysis of expression of the NFκB p50. n=2

FIG. 4: is a Western Blot analysis of expression of the enzyme COX2 and COXIV enzymes in LNCaP prostate cancer cell line after 72 hour incubation with GHRH antagonist JMR-132 and GHRH(1-29)NH_{2.} n=2

FIG. 5: Effects of GHRH and JMR-132 on the protein and lipid oxidation markers as well as on the generation of the ROS in LNCaP prostate cancer cell line.

(A) shows the detection of the expression of the oxidation markers (nitrotyrosine and malondialdehyde) in LNCaP prostate cancer cell line after incubation for 72 hour with GHRH antagonist JMR-132 and GHRH(1-29)NH_{2.} n=2

(B) shows the detection of the expression of the carbonyl groups in LNCaP prostate cancer cell line after 72 hour treatment with GHRH antagonist JMR-132 and GHRH(1-29)NH₂ n=2.

(C) shows the changes in the generation of the reactive oxygen species after 30 minutes incubation. Percentage increase or decrease are expressed vs LNCaP cells cultured in the absence of JMR-132 or GHRH (1-29)NH₂. *** (P<0.005).

FIG. 6: is a Western Blot analysis of expression of SV1 (splice variant 1 of GHRH receptor) and GHRH-R in LNCaP prostate cancer cell line. MCF-7 breast cancer cell line was used as negative control.

FIG. 7: is a Western Blot analysis of expression of the antioxidant enzymes TRX1, NQ01, GPX1 and SOD1 in LNCaP prostate cancer cell line after 72 hour exposure to GHRH antagonist JMR-132 and GHRH(1-29)NH₂ n=2.

### DESCRIPTION OF THE PREFERRED EMBODIMENTS

### Pharmaceutical Compositions and Mode of Administration

The peptides of the invention may be administered in the form of pharmaceutically acceptable, nontoxic salts, such as acid addition salts. Illustrative of such acid addition salts are hydrochloride, hydrobromide, sulphate, phosphate, fumarate, gluconate, tannate, maleate, acetate, trifluoroacetate, citrate, benzoate, succinate, alginate, pamoate, malate, ascorbate, tartarate, and the like. Particularly preferred antagonists are salts of low solubility, e.g., pamoate salts and the like. These exhibit long duration of activity.

The compounds of the present invention are suitably administered to subject humans or animals subcutaneously (s.c.), intramuscularly (i.m.), or intravenously (i.v); intranasally or by pulmonary inhalation; by transdermal delivery; or in a depot form (e.g., microcapsules, microgranules, or cylindrical rod like implants) formulated from a biodegradable suitable polymer (such as D,L-lactide-coglycolide), the former two depot modes being preferred. Other equivalent modes of administration are also within the scope of this invention, i.e., continuous drip, cutaneous patches, depot injections, infusion pump and time release modes such as microcapsules and the like. Administration is in any physiologically acceptable injectable carrier, physiological saline being acceptable, though other carriers known to the art may also be used.

The peptides are preferably administered parenterally, intramuscularly, subcutaneously or intravenously with a pharmaceutically acceptable carrier such as isotonic saline. Alternatively, the peptides may be administered as an intranasal spray with an appropriate carrier or by pulmonary inhalation. One suitable route of administration is a depot form formulated from a biodegradable suitable polymer, e.g., poly-D,L-lactide-coglycolide as microcapsules, microgranules or cylindrical implants containing dispersed antagonistic compounds.

The amount of peptide needed depends on the type of pharmaceutical composition and on the mode of administration. In cases where human subjects receive solutions of GH-RH antagonists, administered by i.m. or s.c. injection, or in the form of intranasal spray or pulmonary inhalation, the typical doses are between 2-20 mg/day/patient, given once a day or divided into 2-4 administrations/day. When the GH-RH antagonists are administered intravenously to human patients, typical doses are in the range of 8-80 μg/kg of body weight/day, divided into 1-4 bolus injections/day or given as a continuous infusion. When depot preparations of the GH-RH antagonists are used, e.g. by i.m. injection of pamoate salts or other salts of low solubility, or by i.m. or s.c. administration of microcapsules, microgranules, or implants containing the antagonistic compounds dispersed in a biodegradable polymer, the typical doses are between 1-10 mg antagonist/day/patient.

### Therapeutic Uses of GH-RH Antagonists.

The most important therapeutic applications of the anti-oxidative action of GH-RH antagonists relate to the redox status of certain cells, including but not limited to cancer cells, reducing the metabolism of reactive oxygen and nitrogen species. This antioxidant activity of GHRH antagonists is employable in the treatment of diseases in which their pathogenesis is related to increased cellular level of oxidative stress. This is of utility with respect to all the neurodegenerative disorders, like Alzheimer's and Parkinson's disease, amyotrophic lateral sclerosis, Huntington's and Alexander's disease as well as inherited ataxias. The redox cellular abnormalities which can also result to cellular protein and lipid damage, are also involved in diabetes and its complications, like macro- and micro-vascular disorders as well as the diabetic neuropathy and diabetic nephropathy.

### EXAMPLES

### Example 1

### Cell Culture and Western Blotting

Prostate cancer cells LNCaP and breast cancer cells MCF-7 were obtained from American Type Culture Collection (Manassas, Va.) and were cultured as described previously [4]. The antibodies that detect P53, PCNA, GPX1, SOD1, NQ01, Thioredoxin 1, COX2 and COX IV were purchased from Cell Signalling (Danvers, Mass.). The antibodies that detect β actin, NFκB50, pNFκB50, caspase 3 and its cleaved form were purchased from Santa Cruz Biotechnology (Santa Cruz, Calif.). The antibodies against GHRH-R (batch number: SV95) and SV1 (batch number: JH 2317/5) were raised in our laboratory. The signals for the immunoreactive proteins were visualized in a Chemi Doc XRS system (Biorad, Hercules, Calif.). The western Blot assay as well the quantification analysis of the blots was performed as described previously[4].

### Example 2

### Detection of Protein and Lipid Oxidation

The detection of the carbonyl groups, the nitrotyrosine and the lipid peroxidation was performed with the Oxiselect Protein carbonyl Immunoblot, the Oxiselect Nitrotyrosine Immunoblot Kit and the Oxiselect Malondialdehyde Immunoblot Kit respectively (Cell Biolabs, San Diego, Calif.) according the manufacturer s instructions. The detection of the lipid peroxidation using a primary rabbit anti-MDA antibody (Cell Biolabs, San Diego, Calif.) according the manufacturer' instructions. The β-actin signal was used as control.

### Example 3

### Measurement of the Intracellular Generation of the Reactive Oxygen Species

The detection of the Reactive Oxygen Species was carried out using aminophenyl fluorescein, an indicator for the highly reactive oxygen species (Invitrogen, Carlsbad, Calif., USA). This fluorescein derivative is non fluorescent until it reacts with the hydroxyl radical, peroxynitrite anion or hypochlorite anion. Upon oxidation, it exhibits green fluorescence which can be detected with a fluorescence plate reader. LNCaP prostate cancer cells were seeded in 200 μl of RPMI 1640 containing 10 μM aminophenyl fluorescein at a density of 10³ cells/well onto a 48-well plate and were incubated for 30 minutes at 37° C. with GHRH (1-29)NH₂ or JMR-132 at a concentration of 10⁻⁶ M. The fluorescence was measured using a fluorescence plate reader (VICTOR³ Multilabel Plate Reader, Perkin Elmer, Shelton, Conn., USA) with an excitation wavelength of 490 nm and an emission wavelength of 515 nm.

### Example 4

### Cell Proliferation Rate Assay and Statistical Analysis

The rate of the cell proliferation was calculated by seeding 10,000 cells in six well plates and after incubation for 4 days counting them under light microscope using the trypan blue assay or a Z series Coulter Counter (Beckman Coulter, Fullerton, Calif., USA). The data are expressed as the mean±SEM. Statistical evaluation of the results was performed by the Student's t test (two-tailed). P values shown are against the control group.

### Results

### Example 5

### The Expression of GHRH Receptor and its Splice Variant 1 in LNCaP Prostate Cancer Cell Line

A band of 45 KDa which reflects the production of GHRH-R [38] as well as a band of 39.5 KDa which is consistent with the size of the SV1 receptor [39] (R.I: 2.37 and 2.90 respectively) were detected in the LNCaP prostate cancer cell line. MCF7 breast cancer cells, which do not express GHRH-R or SV-1 receptor were used as negative control [9] (R.I: 0.06 and 0.08 respectively). The results are shown in Supporting Information (S.I) FIG. S1

### Example 6

### Effect of GHRH(1-29)NH₂ and GHRH Antagonist JMR-132 on the Proliferation Rate and the Expression of the Proliferating Cell Nuclear Antigen (PCNA) in LNCaP Cancer Cells In Vitro

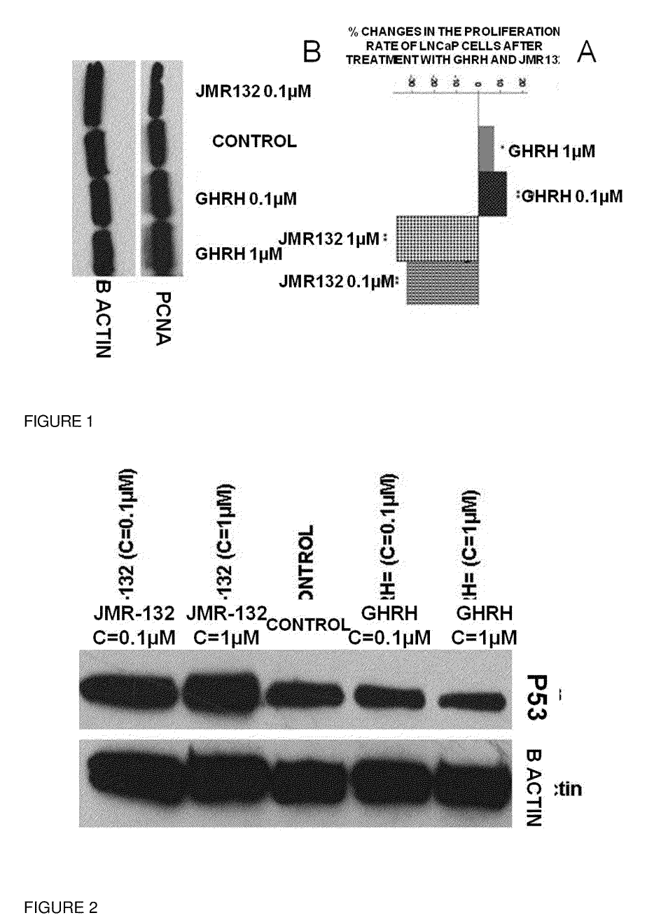

LNCaP prostate cancer cells were exposed to two concentrations of GHRH(1-29)NH₂ and JMR-132. At the dose of 1 μM, GHRH(1-29)NH₂ did not appreciably influence the proliferation rate of the cells, producing an increase of only 7%. However, GHRH (1-29)NH₂ at 0.1 μM concentration stimulated the proliferation rate of LNCaP cells by 13%. GHRH antagonist JMR-132 at the doses of 0.1 μM and 1 μM decreased the proliferation of LNCaP prostate cancer cell line by 32% and 37% respectively. The results are shown in FIG. 1A. In addition, the expression levels of the PCNA (M.W: 36 KDa) were evaluated by Western Blot. The PCNA protein expression was increased in the cells exposed to 0.1 μM and 1 μM of GHRH (1-29)NH₂ (R.I: 0.77 and 0.925 respectively) and decreased in the cells that incubated with 0.1 μM of GHRH antagonist JMR-132. (R.I: 0.495) as compared to control (R.I: 0.656). The results are shown in FIG. 1B.

### Example 7

### Effect of GHRH(1-29 NH₂ and JMR-132 on the Expression of the Wild-Type p53 Tumor Suppressor Protein in LNCaP Cancer Cells In Vitro

LNCaP prostate cancer cell line cultured in vitro was exposed to two concentrations of JMR-132 and GHRH(1-29)NH₂ and the expression level of the p53 tumor suppressor protein (M.W: 53 KDa) was measured by Western Blot. The results are shown in FIG. 2. The p53 protein expression was higher in the cells exposed to 0.1 μM and 1 μM GHRH antagonist JMR-132 (R.I: 0.583 and 0.658 respectively) and lower in the cells incubated with 0.1 μM and 1 μM GHRH (1-29)NH₂ (R.I: 0.376 and 0.264 respectively) as compared to control (R.I: 0.436)

### Example 8

### Effect of GHRH Antagonist JMR-132 and GHRH(1-29)NH₂ on the Expression of NFκB p50 and its Phosphorylated Form, Caspase 3 and the Cleaved Caspase 3 Protein in LNCaP Prostate Cancer Cell Line In Vitro

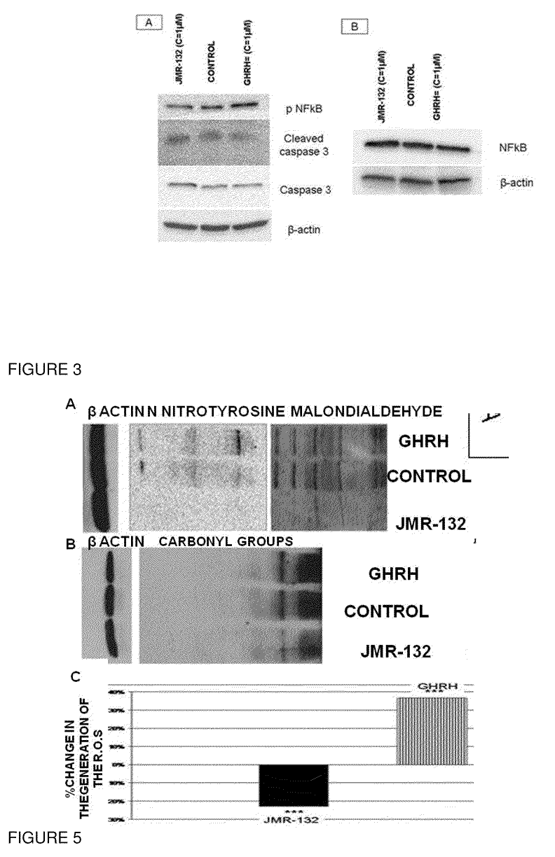

LNCaP cells cultured in vitro were exposed to 1 μM GHRH antagonist JMR-132 and 1 μM GHRH(1-29)NH₂. The expression levels of NFκB p50, the phosphorylated NFκB p50, caspase 3 (M.W: 35 KDa) and the cleaved caspase 3 were detected by Western Blot. The results are shown in FIG. 3A. The expression of the phosphorylated NFκB, caspase 3 protein and its cleaved form was higher in the cells exposed to GHRH antagonist JMR-132 (R.I: 0.451, 0.120, 0.391) and lower in the cells cultured with GHRH (1-29)NH₂ (R.I: 0.623, 0.083, 0.182) as compared to controls (R.I: 0.521, 0.108, 0.320). The expression of the NFκB was not influenced by GHRH (1-29)NH₂ or JMR-132 (R.I: 0.766, 0.786, 0.737. The results are shown in FIG. 3B.

### Example 9

### Effect of JMR-132 and GHRH(1-29)NH₂ on the Expression of the Antioxidant Enzymes GPx1, SOD1, NQ01 and Trx1 in LNCaP Prostate Cancer Cell Line In Vitro

LNCaP prostate cancer cell line cultured in vitro was exposed to 1 μM JMR-132 and GHRH(1-29)NH₂. The expression levels of the detoxifying enzymes were measured by Western Blot. The GPX1 (M.W: 22 Kda) protein expression was higher in the cells exposed to GHRH (1-29)NH₂ (R.I: 0.126) and lower in the cells incubated with GHRH antagonist JMR-132 (R.I: 0.035), as compared to control (R.I:0.107). The SOD1 protein expression (M.W: 18 KDa) was only detectable in the cells that were incubated with GHRH (1-29)NH₂ (R.I:0.111). The NQ01 (M.W: 29 KDa) protein expression was higher in the cells exposed to GHRH (1-29)NH₂ (R.I: 0.196) and much lower in the cells incubated with GHRH antagonist JMR-132 (R.I: 0.025) as compared to control(R.I:0.175). The levels of the thioredoxin 1 protein (M.W: 12 KDa) were elevated in cells treated with GHRH (1-29)NH₂ (R.I: 0.277) and decreased in the cells exposed to JMR-132 (R.I 0.196) as compared to control (R.I: 0.210). The results are shown in Supporting Information (S.I) FIG. S2.

### Example 10

### Effect of GHRH Antagonist JMR-132 and GHRH(1-29)NH₂ on the Expression of the COX 2 and COX IV Enzymes in LNCaP Prostate Cancer Cell Line In Vitro

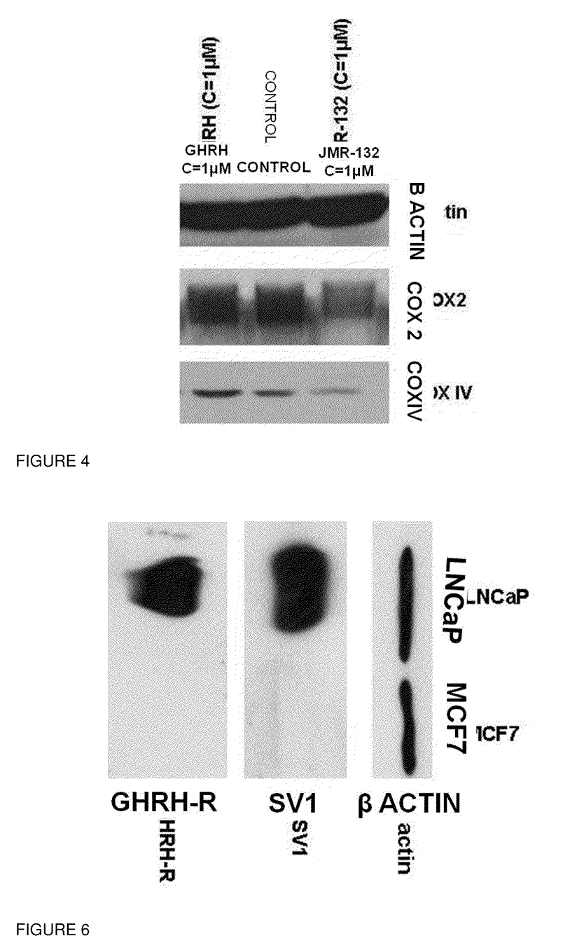

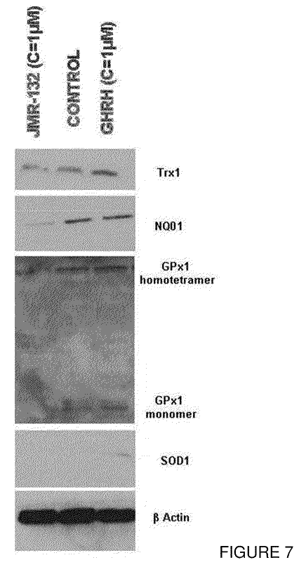

After LNCaP prostate cancer cells cultured in vitro were exposed to 1 μM GHRH antagonist JMR-132 and GHRH(1-29)NH₂, the expression levels of the enzymes COX 2 and COX IV were measured by Western Blot. The COX 2 (M.W: 74 KDa) and COX IV (M.W: 17 KDa) protein expression was higher in the cells exposed to GHRH (1-29)NH₂ (R.I:0.928, 0.237) and lower in the cells incubated with GHRH antagonist JMR-132 (R.I: 0.532, 0.077) as compared to controls (R.I: 0.822, 0.139). The results are shown in FIG. 4.

### Example 11

### Effect of GHRH Antagonist JMR-132 and GHRH(1-29)NH₂ on the Protein and Lipid Oxidation as Well as on the Intracellular Generation of the Reactive Oxygen Species in the LNCaP Prostate Cancer Cell Line In Vitro

LNCaP prostate cancer cell line cultured in vitro were exposed to 1 μM JMR-132 or 1 μM GHRH(1-29)NH₂. The levels of the oxidation of the proteins were determined by the detection of the N-nitrotyrosine and the protein carbonyl groups. Both were elevated in the cells exposed to GHRH (1-29)NH₂ (R.I: 3.282, 7.415) and decreased in the cells incubated with GHRH antagonist JMR-132 (R.I: 1.251, 4.275), as compared to control (R.I: 2.903, 5.846). The results are shown in FIG. 5A. The levels of the lipid peroxidation, determined by the detection of the malondialdehyde (MDA), were increased in cells exposed to GHRH (1-29)NH₂ (R.I:4.89) and decreased in cells incubated with GHRH antagonist JMR-132 (R.I: 2.973) as compared to control (R.I: 4.433). The results are shown in FIG. 5B. In addition, the generation of the reactive oxygen species was higher by 36% in the cells incubated with GHRH (1-29)NH₂ and lower by 23% to cells exposed to JMR-132 as compared to control. The results are shown in the FIG. 5C.

1. A method of reducing the cellular level of oxidative stress in mammals having cells afflicted with said stress, by administering a reductively effective amount of at least one GHRH antagonist whereby said stress is reduced.

2. The method of claim 1 wherein said stress is a function of a neurodegenerative disorder.

3. The method of claim 2 wherein said neurodegenerative disorder is selected from the group consisting of Alzheimer's and Parkinson's disease, amyotrophic lateral sclerosis, Huntington's and Alexander's disease and inherited ataxias.

4. A method of reducing the metabolism of reactive oxygen and nitrogen species in cancer cells in mammals by administering a reductively effective amount of at least one GHRH antagonist whereby said metabolism is reduced.
